(19) United States
(12) Patent Application Publication
(10) Pub. No.: US 2011/0256222 A1
(43) Pub. Date: Oct. 20, 2011

(54) Recombinant Protein Enriched in a Heparin Binding Site and/or in a Heparan Sulfate Binding Site

(76) Inventors: Arjo Lysander De Boer, Tilburg (NL); Jan Bastiaan Bouwstra, Tilburg (NL)

(21) Appl. No.: 12/936,739

(22) PCT Filed: Apr. 7, 2009
(86) PCT No.: PCT/NL09/50179

§ 371 Date: Oct. 7, 2010

(30) Foreign Application Priority Data
Apr. 10, 2008 (EP) 08154329.0

(51) Int. Cl. A61K 9/00; A61K 38/17; A61K 35/00; A61P 25/00; A61P 9/00; A61P 19/00; A61P 17/02; C07K 14/00; C12P 21/06

(52) U.S. Cl. 424/484; 530/354; 514/17.2; 424/93.1; 435/69.1

(57) ABSTRACT

The invention relates to a recombinant protein enriched in a heparin binding site and/or a heparan sulfate binding site. Such recombinant protein is used as an in vivo controlled release system of a protein of interest.

### FIELD OF THE INVENTION

The invention relates to a recombinant protein that is enriched in a heparin and/or a heparan sulfate binding sites (HBSs). The invention also relates to a controlled release system that comprises such a recombinant protein, a heparin and/or a heparan sulfate and a protein of interest and to their use.

### BACKGROUND OF THE INVENTION

Several delivery systems for proteins and growth factors are already known. Some of these delivery systems are only able to present and deliver peptides (Schense J. C. et al, (1999), Bioconj. Chem., 10:75-81). While peptides can partially mimic the bioactivity of the whole protein from which they are derived, this bioactivity is usually lower than the bioactivity of the whole protein, and sometimes it is impossible to mimic certain proteins with only a short peptide. It would therefore be desirable to be able to incorporate the entire protein, such as a growth factor or other pharmaceutically active molecule, into the matrix.

The need is particularly great for delivery systems that could locally present any protein of interest, wherein the protein of interest retains its in vivo activity, is protected from any type of degradation and is released in a controlled way.

WO 01/83522 discloses a delivery system, wherein a protein of interest is incorporated into a protein or polysaccharide matrix. In one embodiment, heparin is bound to the matrix to form a heparin-matrix. A protein of interest is engineered to bind the heparin-matrix. The protein of interest is released from the heparin-matrix upon cleavage of the site between the protein and heparin and/or degradation of the matrix. Delivery systems disclosed in WO 01/83522 are quite complicated and therefore laborious and expensive to prepare since several functional domains need to be present. In WO 01/83522 heparin is non-covalently attached to fibrin gels using a two-part system consisting of a peptide chimera and heparin itself. The peptide chimera consists of two domains, a factor XIIIa substrate and a polysaccharide-binding domain. Once the peptide chimera is cross-linked into the fibrin gel, it attaches the heparin (or other polysaccharides) by non-covalent interactions.” This means that 4 components (matrix, chimera, heparin and protein of interest) need to be produced separately and combined afterwards to create a controlled release composition. The invention as described herein allows production of a matrix able to bind heparin excluding the need of the chimera as described in WO 01/83522.

Therefore, there is still a need is for a more simple delivery system that could be used to deliver any protein of interest wherein said protein of interest retains its in vivo activity, is protected from any type of degradation and is released in a controlled way.

### DESCRIPTION OF THE INVENTION

### Recombinant Protein

In a first aspect, there is provided a recombinant protein enriched in a heparin binding site and/or a heparan sulfate binding site (HBS).

A recombinant protein may be any protein. In one embodiment, it is an active ingredient, which may be used itself as a therapeutical agent to prevent, treat, delay any type of disease or conditions. A therapeutical agent may be for the treatment of pain, cancer, a cardiovascular disease, myocardial repair, angiogenesis, bone repair and regeneration, wound treatment, neural stimulation/therapy or diabetics.

In a preferred embodiment, a protein does not already contain a heparin and/or a heparan sulfate binding site in its sequence. In this embodiment, a protein is modified to comprise at least one, at least two, at least three, at least four, at least five, at least six, at least seven, at least eight, at least nine, at least ten or more heparin binding sites and/or heparan sulphate binding sites.

In another preferred embodiment, a protein already comprises a heparin and/or a heparan sulfate binding site. In this preferred embodiment, a recombinant protein does not necessarily need to be modified to comprise a heparan or heparan sulfate binding site. Alternatively, according to a more preferred embodiment, a recombinant protein of the invention comprises a protein of interest which already comprises a heparin or heparan sulfate binding site is modified to comprise at least one, at least two, at least three, at least four, at least five, at least six, at least seven, at least eight, at least nine, at least ten or more additional Heparin binding sites and/or additional heparan sulfate binding sites compared to the protein it derived from. Such a protein may be seen as a recombinant protein. Examples of proteins comprising a heparin and/or a heparan sulfate binding site are growth factors that are involved in morphogenesis, both in the developing organism and in the adult, and that are known to bind extracellular matrix molecules. Growth factors that bind heparin include the transforming growth factor (“TGF”)-betasuperfamily (including the bone morphogenic proteins, “BMPs”), the fibroblast growth factor (“FGF”) family (Presta, M., et al., (1992). Biochemical and Biophysical Research Communications. 185:1098-1107), and vascular endothelial growth factor (“VEGF”), among others. Preferred examples are bone morphogenetic factor-2 (BMP-2) and fibroblast growth factor 2 (FGF-2). Additional “heparin-binding” protein include interleukin-8, neurotrophin-6, heparin-binding epidermal growth factor, hepatocyte growth factor, connective tissue growth factor, midkine, and heparin-binding growth associated molecule. These proteins have been shown to regulate tissue repair (Gotz, R., et al, (1994). Nature. 372:266-269; Kaneda, N., et al, (1996) J Biochem. 119:1150-1156; Kiguchi, K., et al, (1998) Mol. Carcinogensis. 22:73-83; Kinosaki, M., et al, (1998). Biochim. Biophys. Acta. 1384:93-102; McCaffrey, T., et al, (1992) J. Cell. Physiol. 152:430-440; Nolo, R., et al, (1996) Eur. J. Neurosci. 8:1658-1665; Spillmann, D., et al, (1998). Journal of Biological Chemistry. 273: 154815493; Steffen, C., et al, (1998); Growth Factors. 15:199-213. Tessler, S., et al, (1994) J; Biol. Chem. 269:12456-12461). These proteins have shown the potential to enhance healing in many different types of tissue including vasculature, skin, nerve and liver. Therefore, these proteins can be used to enhance wound healing in many different parts of the body by selecting a given protein and by enriching it with a heparin and/or a heparan binding site as described herein.

Alternatively, a preferred recombinant protein is a protein which is not known as a therapeutic agent. In this embodiment, a recombinant protein is preferably at least one of non-immunogenic, neutral and biodegradable. More preferably, a recombinant protein is a recombinant gelatine-like protein as later defined herein.

### Heparin

“Heparin” (also referred to a heparinic acid) is a heterogenous group of highly sulfated, straight-chain anionic mucopolysaccharides, called glycosaminoglycans. Although others may be present, the main sugars in heparin are: alpha.-L-iduronic acid 2-sulfate, 2-deoxy-2-sulfamino-alpha.-glucose 6-sulfate, beta.-D-glucuronic acid, 2-acetamido-2-deoxy-alpha.-D-glucose, and L-iduronic acid. These and optionally other sugars are joined by glycosidic linkages, forming polymers of varying sizes. Due to the presence of its covalently linked sulfate and carboxylic acid groups, heparin is strongly acidic.

The molecular weight of heparin varies from about 6,000 to about 20,000 Da, or from 6,000 to 20,000 Da depending on the source and the method of determination. Native heparin is a constituent of various tissues, especially liver and lung, and mast cells in several mammalian species. Heparin and heparin salts (heparin sodium) are commercially available and are primarily used as anticoagulants in various clinical situations.

### Heparin or Heparan Sulfate Binding Site

As indicated in a previous paragraph, a heparin and/or a heparan sulfate binding site may be either already present in a protein and may be incorporated into a protein sequence. Throughout this application, the expression heparin binding site is synonymous with heparan sulfate binding site. In a preferred embodiment, a heparin and/or a heparan sulfate binding site is incorporated into a protein sequence. Several heparin binding sites sequences are known to the skilled person. WO 01/83522 exemplifies several heparin binding sites in table 2 at page 10 of the published application. One may exemplifies: K(A)FAKLAARLYRKA (from anti-thrombin III), YKKIIKKL (from Platelet factor 4), KHKGRDVILKKDVR (from neural cell adhesion molecule), YEKPGSPPREVVPRPRPCV and KNNQKSEPLIGRKKT (from fibronectin), KDPKRL and YRSRKY (from bFGF basic fibroblast growth factor), YKKPKL (from aFGF acidic fibroblast growth factor), AKRSSKM and CRKRCN (from LPL lipoprotein lipase), GBBGB, GLPGMKGHRGFS, GRKGR, GKRGK and KEDK, wherein B is a basic amino acid. In a more preferred embodiment, a heparin or a heparan sulfate binding site is selected from the following group consisting of: GBBGB, GLPGMKGHRGFS, GRKGR, GKRGK and KEDK, wherein B is a basic amino acid. Examples of basic amino acids are: histidine, lysine and arginine. In an even more preferred embodiment, a heparin or a heparan sulfate binding site is selected from the following group consisting of: GBBGB, GLPGMKGHRGFS, GRKGR, and GKRGK wherein B is a basic amino acid. Examples of basic amino acids are: histidine, lysine and arginine. In an even more preferred embodiment, a heparin or heparan sulfate binding site is GLPGMKGHRGFS. This HBS is preferred because this HBS fits also the GXY format of a gelatine like structure as defined later herein. Most preferably, it is GLPGMKGHRGFS wherein the last S has been removed, since it is a potential glycosylation target, which may interfere with heparin/heparan sulfate binding: GLPGMKGHRGF.

As used herein “heparin-binding”, refers to the ability of a molecule to bind with heparin or heparin sulfate, as determined by direct or indirect heparin-binding assays known in the art, such as the affinity co-electrophoresis (ACE) assay for peptide-glycosaminoglycan binding as described in WO2005014619

A recombinant protein of the invention is also preferably named a recombinant protein enriched in HBS or a HBS-enriched protein. The term “HBS-enriched” (or enriched in a Heparin Binding Site and/or in a heparan sulfate binding site) refers herein to an amino acid sequence of a protein comprising at least one HBS motif as defined above. The term “HBS-enriched” in the context of this invention preferably means that a certain level of a HBS motif, calculated as a percentage of the total number of amino acids per protein and that there is a certain even distribution of HBS motifs or sequences in an amino acid sequence of a protein. The level of HBS sequences is expressed as a percentage. This percentage is calculated by dividing the total number of amino acids that constitute a given HBS motif by the total number of amino acids of a protein and multiplying the result with 100.

More preferably, “HBS-enriched” refers herein to an amino acid sequence of a protein wherein the percentage of a HBS motif related to the total number of amino acids of a protein is at least 1.5 and if the amino acid sequence comprises 350 amino acids or more, each stretch of 350 amino acids contains at least one HBS motif. Preferably the percentage of a HBS motif is at least 2.0, more preferably at least 2.5, more preferably at least 3.0, more preferably at least 3.5 and most preferably at least 4.5.

The terms “HBS sequence” and “HBS motif” are used interchangeably.

A recombinant protein enriched in HBS is expected to have improved ability to bind heparin and/or heparan sulfate. Improved preferably means that a protein of the invention hence modified has an ability to bind heparin and/or heparan sulfate which is at least 5% increased by comparison to the corresponding protein which has not been modified according to the invention. Preferably, the increase is of at least 7%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200% or more. In another preferred embodiment, the improvement is by comparison to a control protein which is known not to be able to bind heparin and/or heparan sulfate. A preferred control protein is a gelatine-like protein P4 disclosed in EP 1 014 176. The assessment of the binding to heparin and/or heparan sulfate has already been described herein.

### Composition and Matrix

In a further aspect, there is provided a composition comprising a recombinant protein as defined in the previous section. Preferably, a composition additionally comprises a heparin and/or a heparan sulfate. Accordingly, in a preferred embodiment, there is provided a composition further comprising a heparin and/or a heparan sulphate, preferably in which the heparin and/or heparin sulphate is from a non-mammalian source.

In a further aspect, there is provided a matrix comprising a composition as defined in the previous aspect. A matrix according to the invention is a three dimensional network comprising biocompatible polymers that are co-valently or non co-valently cross/linked. Several preferred ways of cross/linking are exemplified in the general definitions. The biocompatible polymers can be selected from, but not limited to: polystyrenes, polyphosphoester, polyphosphazenes, aliphatic polyesters, poly 3-hydroxybutyric acid, polylactic acid, polyethylene glycol polyvinyl alcohol, polyacrylamide or polyacrylic acid, glycosaminoglycans such as hyaluronic or chitosan acid and the like modified polysaccharides such as cellulose or starch, or polypeptides as poly-1-lysine more preferably extracellular matrix proteins like gelatins, collagens, elastin, or fibrin and the like or recombinant gelatine-like proteins or recombinant collagen-like proteins. In a preferred embodiment the biocompatible polymers of the matrix comprises a recombinant protein as defined in the previous section.

In a preferred embodiment, a matrix as defined herein is from a non-mammalian source. More preferably a heparin or heparan sulfate is from the company Bio Tie (http://www.biotie.fi/page/en/research/trombosis/bioheparin.).

In a further preferred embodiment, a composition or a matrix as earlier defined herein additionally comprises a protein of interest and/or a cell. A protein of interest may be a native protein isolated from a natural source or a recombinantly produced protein of interest. Preferably, a protein of interest is considered as a recombinantly produced protein. In a more preferred embodiment, a protein of interest and/or a cell comprises a heparin and/or a heparan sulfate binding site. A protein of interest may already comprise such a site. A cell may already comprise a heparin and/or a heparin sulphate binding site such as a receptor recognizing a heparin and/or a heparin sulphate. For example a membrane bound epidermal growth factor is known to recognize and/or bind a heparin and/or a heparin sulfate (Takamura et al. J Biol. Chem. 1997 Dec. 5; 272(49):31036-42 or Bertolesi et al. J Biol Regul Homeost Agents. 2005 January-June; 19(1-2):33-40).

Accordingly, in a preferred embodiment a system is provided comprising a matrix as defined herein. This system may be called a controlled release system for a protein of interest and/or a cell. Said system comprises a matrix, a protein of interest and/or a cell. This system may also be called a cell support system comprising a matrix as defined herein.

Alternatively or in combination with the previous embodiments, a recombinantly produced protein of interest may be modified to introduce one or multiple heparin and/or heparan binding sites. The introduction of an heparin and/or heparan binding site in a protein of interest may be carried out the same way as for the introduction of such a site in a recombinant protein as already defined herein. The number of heparin binding sites and/or heparin sulphate binding sites is at least one.

All kinds of proteins of interest or pharmaceutical agents can be used in the matrix or composition. The term “pharmaceutical” refers to chemical or biological molecules providing a therapeutic, diagnostic, or prophylactic effect, preferably in vivo. The term pharmaceutical is also meant to indicate prodrug forms thereof. A “prodrug form” of a pharmaceutical means a structurally related compound or derivative of the pharmaceutical which, when administered to a host is converted into the desired pharmaceutical. A prodrug form may have little or none of the desired pharmacological activity exhibited by the pharmaceutical to which it is converted. Examples of proteins which can be incorporated into a composition or matrix of the present invention include, but are not limited to, hemoglobin, vasporessin, oxytocin, adrenocorticocotrophic hormone, epidermal growth factor, prolactin, luliberin or luteinising hormone releasing factor, human growth factor, basic fibroblast growth factor, hepatocyte growth factor, angiogenesis growth factor, vascular endothelial growth factor, bone morphogenetic growth factor, nerve growth factor, and the like; interleukins; enzymes such as adenosine deaminase, superoxide dismutase, xanthine oxidase, and the like; enzyme systems; blood clotting factors; clot inhibitors or clot dissolving agents such as streptokinase and tissue plasminogen activator; antigens for immunization; hormones. A protein of interest as defined herein may be any protein, therefore it may be identical with a recombinant protein as earlier defined herein. However, in a preferred embodiment, a protein of interest is distinct from a recombinant protein as defined herein. A protein of interest preferably used in the invention natively comprises a heparin and/or a heparan sulphate binding site. These proteins are involved in morphogenesis, both in the developing organism and in the adult, and that are known to bind extracellular matrix molecules. Growth factors that bind heparin include the transforming growth factor (“TGF”)-betasuperfamily (including the bone morphogenic proteins, “BMPs”), the fibroblast growth factor (“FGF”) family (Presta, M., et al., (1992). Biochemical and Biophysical Research Communications. 185:1098-1107), and vascular endothelial growth factor (“VEGF”), among others. Preferred examples are bone morphogenetic factor-2 (BMP-2) and fibroblast growth factor 2 (FGF-2). Additional “heparin-binding” protein include interleukin-8, neurotrophin-6, heparin-binding epidermal growth factor, hepatocyte growth factor, connective tissue growth factor, midkine, and heparin-binding growth associated molecule. These proteins have been shown to regulate tissue repair (Gotz, R., et al, (1994). Nature. 372:266-269; Kaneda, N., et al, (1996) J. Biochem. 119:1150-1156; Kiguchi, K., et al, (1998) Mol. Carcinogensis. 22:73-83; Kinosaki, M., et al, (1998). Biochim. Biophys. Acta. 1384:93-102; McCaffrey, T., et al, (1992) J. Cell. Physiol. 152:430-440; Nolo, R., et al, (1996) Eur. J. Neurosci. 8:1658-1665; Spillmann, D., et al, (1998). Journal of Biological Chemistry. 273:154815493; Steffen, C., et al, (1998); Growth Factors. 15:199-213. Tessler, S., et al, (1994) J; Biol. Chem. 269:12456-12461). These proteins have shown the potential to enhance healing in many different types of tissue including vasculature, skin, nerve and liver. Therefore, these proteins can be used to enhance wound healing in many different parts of the body by selecting a given protein.

The skilled person will understand that the amount of each of the constituents of a composition and matrix as defined herein may have to be adjusted depending on the application envisaged. In a preferred embodiment, an amount of heparin ranged between about 20 μg and about 1.5 mg together with between about 5 μg and about 50 μg of protein of interest is being administered. In another preferred embodiment, an amount of heparin ranged between 20 μg and 1.5 mg together with between 5 μg and 50 μg of protein of interest is being administered. Very good results were obtained with 26 μg and 1 mg heparine together with 20 μg of a protein of interest.

Optionally, a composition or matrix may comprise adjuvants like buffers, salts, surfactants, humectants and co-solvents. Also the amount and kinds of used pharmaceuticals can be varied, for example the use of at least two proteins of interest or the use of a protein of interest in combination with an antibiotic.

A composition or a matrix as defined herein is particularly advantageous since this constitutes a controlled released system of a protein of interest and/or of a cell once a composition or a matrix has been introduced into a subject. Heparin and/or heparan sulfate will bind to and/or target a protein of interest and/or a cell via a heparin or heparan sulfate binding site present in a recombinant protein and optionally in a protein of interest and/or a cell. As a consequence, a protein of interest and/or a cell will not freely diffuse from the composition. A protein of interest and/or a cell will be subsequently released upon in vivo degradation of the matrix. The time course of this release depends on the identity of the biocompatible polymers of the matrix. Alternatively or in combination with previous embodiments if a matrix comprises a recombinant protein, as biocompatible polymer one may influence the duration of the release of a protein of interest and/or a cell by incorporating one or more cleavage sites in the recombinant protein present in the matrix. For example, the duration of the release of a protein of interest may vary from, approximately one week and up to approximately several months or more, or from one week and up to several months or more.

A cleavage site may be an enzymatic cleavage site (either proteolytic or polysaccharide degrading), or a site which is cleavable by non-specific hydrolysis (i.e. an ester bond). A cleavage site allows the engineering of more specific release of a protein of interest from a matrix comprising a recombinant protein. A cleavage site also allows a protein of interest to be released with little or no modification to its primary protein sequence, which may result in higher activity of a protein of interest. In addition, it allows the release of a protein of interest to be even more controlled by cell specific processes, such as localized proteolysis, rather than only by metabolisation of a recombinant protein. Enzymes that could be used for proteolytic degradation are numerous. Proteolytically cleavage sites could include substrates for collagenase, plasmin, elastase, stromelysin, or plasminogen activators (as exemplified in WO 01/83522). Enzymatic degradation may also occur with a polysaccharide substrate for enzymes such as heparinase, heparitinase, and chondroitinase ABC. Each of these enzymes have polysaccharide substrates.

Enzymatic degradation may also occur with a protease. A proteolytic degradation sequence may be inserted, such the one of plasmin. Non-enzymatic cleavage may consist of any linkage which undergoes non-specific hydrolysis by an acid or base catalyzed mechanism. These substrates can include oligo-esters such as oligomers of lactic or glycolic acid. The rate of degradation of these materials can be controlled through the choice of an oligomer.

In another embodiment, a matrix as defined herein constitutes a cell support. In this preferred embodiment, a composition comprises at least one recombinant protein and a heparine and/or a heparan sulfate. A matrix according to this embodiment is advantageous as a cell support as cells can attach to it via heparine binding receptors on their cell surface, for example but not limited to membrane bound epidermal growth factor as earlier identified herein. In another preferred embodiment, a matrix which constitutes a cell support comprises at least one recombinant protein, a heparine and/or a heparan sulfate and a protein of interest. A matrix according to this embodiment is particular advantageous as a cell support because growth promoting agents or growth factors already described herein can be incorporated into the matrix to guide cells or to promote cell-growth or cell differentiation. Cell supports comprising a composition according to the invention, on which cells have been grown can be applied during, for example, transplantation of skin or wound treatment or to enhance bone or cartilage (re)growth. It is also possible to coat implant materials with a composition of the invention to activate and adhere cells in vivo which promote implantation.

### Nucleic Acid Molecule

In a further aspect, there is provided a nucleic acid molecule represented by a nucleic acid sequence encoding a recombinant protein and/or a protein of interest both enriched in HBS as defined in the previous section. As already stated herein, in a preferred embodiment, a recombinant protein is distinct from a protein of interest. Therefore, a nucleic acid sequence encoding a recombinant protein is preferably distinct from a nucleic acid sequence encoding a protein of interest. The preparation of a nucleic acid molecule of the invention is carried out using molecular biology techniques known to the skilled person (Molecular Cloning: A Laboratory Manual 3^{rd} ed. by J. Sambrook and David W. Russell; January 2001, Cold Spring Harbor Laboratory Press)

### Nucleic Acid Construct or Expression Vector

In a further aspect, there is provided a nucleic acid construct or expression vector comprising a nucleic acid molecule as defined in the previous section. Optionally, a nucleic acid sequence present in said nucleic acid construct is operably linked to one or more control sequences, which direct the production of a recombinant protein or of a protein of interest both enriched in HBS in a suitable expression host.

The invention also relates to an expression vector comprising said nucleic acid construct of the invention. Preferably, an expression vector comprises a nucleic acid sequence of the invention, which is operably linked to one or more control sequences, which direct the production of an encoded recombinant protein and/or a protein of interest both enriched in HBS in a suitable expression host. At a minimum control sequences include a promoter and transcriptional and translational stop signals. An expression vector may be seen as a recombinant expression vector. An expression vector may be any vector (e.g. plasmic, virus), which can be conveniently subjected to recombinant DNA procedures and can bring about the expression of a nucleic acid sequence encoding a recombinant protein and/or a protein of interest both enriched in HBS. Depending on the identity of the host wherein this expression vector will be introduced and on the origin of the nucleic acid sequence of the invention, the skilled person will know how to choose the most suited expression vector and control sequences.

### Host Cell

In yet a further aspect, there is provided a host cell or cell or host comprising a nucleic acid construct or expression vector as defined in the previous section.

Suitably a host cell is a expression host cell like Hansenula, Trichoderma, Aspergillus, Penicillium, Saccharomyces, Kluyveromyces, Neurospora or Pichia. Fungal and yeast cells are preferred to bacteria as they are less susceptible to improper expression of repetitive sequences. Methylotrophic yeast hosts are more preferred. Examples of methylotrophic yeasts include strains belonging to Hansenula or Pichia species. Preferred species include Hansenula polymorpha and Pichia pastoris. More preferably, a host will not have a high level of a protease or a proteolytic enzyme that could have attacked or degraded a protein of interest when expressed. Most preferably, a host has been modified to be deficient in the expression of a protease and/or a proteolytic enzyme and/or any other undesirable protein. In this respect, Pichia or Hansenula offers an example of a very suitable expression system. Use of Pichia pastoris as an expression system is disclosed in EP-A-0926543 and EP-A-1014176. In one embodiment, a host is free of active post-translational processing mechanism such as in particular hydroxylation of proline (i.e. it lacks a functional prolyl-4-hydrolase) and also hydroxylation of lysine. A host lacking a functional prolyl-4-hydrolase will express a gelatine-like protein monomer or multimer having less than 10%, more preferably less than 5%, less than 4%, less than 3 or 2%, most preferably less than 1% of the proline residues of the GXY triplets and/or of the total proline residues in the polymer are hydroxylated. In another embodiment, a host has an endogenic proline hydroxylation activity by which a recombinant gelatine-like protein is hydroxylated in a highly effective way. The selection of a suitable host cell from known industrial enzyme producing fungal host cells specifically yeast cells on the basis of the required parameters described herein rendering a host cell suitable for expression of recombinant gelatine-like proteins suitable to be used according to the invention in combination with knowledge regarding the host cells and the sequence to be expressed will be possible by a person skilled in the art.

### Production Method

In yet a further aspect, there is provided a method for the production of a recombinant protein and/or a protein of interest both enriched in HBS as defined in a previous section. In this method, preferably a host cell as defined in a previous section is cultured under suitable conditions leading to expression of a recombinant protein and/or a protein of interest, and subsequently optionally a recombinant protein and/or a protein of interest is recovered from a host cell.

A preferred method for producing a recombinant protein and/or a protein of interest according to present invention comprises:
preparing an expression vector comprising a nucleic acid sequence encoding a recombinant protein and/or a protein of interest as defined in a previous section,
expressing said nucleic acid sequence in a host, preferably a yeast, more preferably a methylotrophic yeast,
culturing said yeast under suitable fermentation conditions to allow expression of said nucleic acid sequence;
optionally purifying said recombinant protein and/or protein of interest from the culture.

A recombinant protein of the invention comprising a gelatin-like protein may be produced by recombinant methods as disclosed in EP-A-0926543, EP-A-1014176 or WO01/34646. Also for enablement of the production and purification of a recombinant protein of the invention comprising a gelatin-like protein reference is made to the examples in EP-A-0926543 and EP-A-1014176 wherein Pichia pastoris is used as host cell.

A recombinant protein and a protein of interest as defined herein may be both produced in the same host cell and in one single method (simultaneous production of both proteins). Alternatively a recombinant protein and a protein of interest may be produced in identical or distinct host cells in two distinct methods.

### Medical Use

In a further aspect, there is provided a composition or a matrix all as defined herein for use as a therapeutic agent. A composition or a matrix as defined herein is a controlled release system for a protein of interest and/or a cell or is a cell support once it has been introduced into a subject. A therapeutical agent or pharmaceutical agent may be a protein of interest and/or a cell. Several pharmaceutical agents have been already identified herein. The skilled person will understand that the invention is not limited to a specific type of therapeutic agent. The controlled release system of the invention could potentially be used for any protein of interest which is known to be used as a therapeutical agent or which could potentially be used as a therapeutical agent.

In a preferred embodiment, a therapeutical agent is for promoting cell repair, regeneration or remodeling or inhibiting cell repair or regeneration or for the treatment of pain, cancer therapy, cardiovascular diseases, myocardial repair, angiogenesis, bone repair and regeneration, wound treatment, neural stimulation/therapy or diabetics. A cell as part of a matrix as defined herein may for example be used in a therapeutic application to provide insulin into the pancreas of subject suffering from diabetes type I.

The skilled person will understand that the invention further relates to a further aspect wherein there is provided the use of a composition or a matrix for the manufacture of a therapeutical agent for the prevention or treatment of any of the conditions or diseases as identified herein.

A therapeutical agent as used herein may further comprise one or more additional therapeutical agent or active ingredient. A therapeutical agent as used herein may be administered by injection (subcutaneous, intravenous or intramuscular) or orally or via inhalation. However, a therapeutical agent as used herein may also be implanted via surgery. Yet another suitable route of administering is via an external wound dressing or even transdermally.

### Method

In a further aspect, there is provided a method for introducing a composition and/or a matrix all as defined herein in a subject in a need thereof wherein a protein of interest and/or a cell is released in said subject. Preferably, a protein of interest and/or a cell are released in said subject (in vivo) from said composition and/or matrix.

### General Definitions

In a preferred embodiment, a recombinant protein as used herein is a gelatine-like protein as defined below.

### Gelatine-Like Protein as an Example of a Recombinant Protein

A gelatine-like protein is preferably a multimer or a multimeric peptide that comprises or consists of at least 2, 3, 4, 5, 6, 7, 8, 9 or 10, 11, 12 repeats of a monomer. Several preferred monomers are identified further herein.

A gelatine-like protein may be a gelatine-like protein monomer (or a polymer comprising or consisting of monomers) preferably comprises a substantial number, or consists of, GXY triads, wherein G is Glycine and X and Y are any amino acid. A substantial number of GXY triads refers to at least about 50%, or at least 50%, more preferably at least 60%, 70%, 80%, 90% or most preferably 100% of amino acid triplets of a whole gelatin-like protein monomer being GXY, especially consecutive GXY triplets. The N- and/or C-terminal end of a monomer and/or polymer may comprise other amino acids, which need not be GXY triplets. Also, the molecular weight of the monomer is preferably at least about 15 kDa (calculated molecular weight), more preferably at least about 16, 17, 18, 19, 20, 30, 40, 50, 60, 70, 80, 90 kDa or more, or preferably at least 15 kDa (calculated molecular weight), more preferably at least 16, 17, 18, 19, 20, 30, 40, 50, 60, 70, 80, 90 kDa or more.

Whereas often the terms ‘collagen’, ‘collagen-related’, ‘collagen-derived’ or the like are also used in the art, the term ‘gelatine’ or ‘gelatine-like’ protein may also be used. Natural gelatine is a mixture of individual polymers with MW's ranging from 5,000 up to more than 400,000 daltons. “Native” or “natural” collagens or collagenous domains refer to those nucleic acid or amino acid sequences found in nature, e.g. in humans or other mammals. In an embodiment, a gelatine-like protein is a recombinant protein.

The expressions “triple helical domain” and “collagenous domain” are used interchangeably, and refer to the Gly-Xaa-Yaa triplet repeat region (or GXY triads) or motif of the recombinant or natural collagen, i.e. [Gly-Xaa-Yaa]n, wherein Xaa and Yaa are any amino acid and wherein n is at least 5, 10, 15, 20, 30, 40, 50, 70, 80, 90, 100 or more. For example in natural human COL1A1 as depicted in SEQ ID NO: 1, the collagenous domain is from amino acid 179 to amino acid 1192, the whole of which or a variant or a fragment of which (or of the variant) may be used herein. A gelatine-like protein means either a gelatine-like protein monomer or a gelatine-like protein multimer.

It is a further embodiment that a recombinant gelatine-like protein used herein does not contain any S (Ser) and/or T (Thr) and/or N (Asn). Thus, for example S and/or T and/or N found in natural collagen domains (or fragments thereof) may be replaced and/or deleted using known molecular biology methods. Alternatively, natural fragments may be selected which are free of S and/or T and/or N. Glycosylation usually takes place at these amino acids: Asn (N-glycosydic structures), Ser or Thr (O-glycosidic structures). Glycosylation is thereby reduced or prevented, which is an advantage for applications where no immune response is desired.

In a further preferred embodiment, a gelatine-like protein used is in essence free of proline residues. Even more preferably, free of hydroxyproline residues. Hydroxylation is a requirement for the formation of triple helices in collagen and plays a role in gelation of gelatine. Accordingly, in a preferred embodiment, a gelatine-like protein is essentially free of glycosylation and/or essentially free of hydroxyproline residues. In this context, “essentially free” may mean that a gelatine-like protein may contain one or no glycosylation sites and/or one or no hydroxyproline residues. More preferably, a gelatine-like protein is free of glycosylation and/or free of hydroxyproline residues.

A gelatine-like protein as used herein may also be a multimer of a monomer of the sequence as described herein. Therefore, in a further embodiment a recombinant gelatine-like protein is provided comprising or consisting of a multimer of a monomer sequence described above. Preferably, a monomer repeat is a repeat of a same monomer unit (having identical amino acid sequences), although optionally combinations of different monomer units (having different amino acid sequences, each falling under the criteria above) may be used. Preferably, monomer units are not separated by spacing amino acids, although short linking amino acids, such as 1, 2, 3, 4 or 5 amino acids, may also be inserted between one or more of the monomers.

In one embodiment, a multimer comprises or consists of at least 2, 3, 4, 5, 6, 7, 8, 9 or 10, 11, 12 repeats of a monomer as described above or more, e.g. of a sequence substantially identical to amino acids 179-1192 of SEQ ID NO: 1.

Thus, a recombinant polymer may comprise n monomer units, each monomer fulfilling the above criteria, wherein n is the number of monomer repeats that is required to build a multimer of about 15 to 150 kDa. The value of n therefore depends on the size of the monomer. For a monomer of 10 amino acids, n may for example be 10 to 100, or more. For a monomer with 100 amino acids n may e.g. be 1 to 10. A polymer may comprise identical or different monomer units, linked consecutively. Each monomer preferably comprises at least one XRGD, wherein X is not D or P or O. As the monomers are free of DRGD and/or PRGD, the polymer is also free of these motifs. Preferably, a polymer comprises at least n XRGD motifs (wherein X is not D, P or O) and no DRGD, PRGD or ORGD motifs. Preferably a polymer is also free of S, T and/or N. Accordingly, a preferred recombinant gelatine-like protein is a multimeric peptide or a polymer comprising at least one XRGD wherein X is not D, P or O.

The term “improved stability” means that an XRGD- or RGD-enriched gelatine is not hydrolysed or is hydrolysed to a lesser extent, preferably by at least a factor 2, under usual culture conditions of the yeast expression host compared to the corresponding sequence having DRGD, PRGD or ORGD (O meaning hydroxyproline).

In a further preferred embodiment, a gelatine-like protein being used as a recombinant protein has been enriched in a heparin binding site comprises as monomer SEQ ID No.:2 as described in the example. This is a monomer of HBC (Heparin Binding Collagen like polypeptide) which has been enriched for heparin binding sites. The preparation of this recombinant protein has been extensively described in the example. Multimers comprising or consisting of at least 2, 3, 4, 5, 6, 7, 8, 9 or 10, 11, 12 repeats of this monomer of SEQ ID NO:2 may be prepared the same way as for multimers of SEQ ID NO: 1. All definitions that have been given for a multimer comprising as monomer SEQ ID NO:1 also hold for a multimer comprising as monomer SEQ ID NO:2.

A “fragment” is a part of a longer nucleic acid or polypeptide molecule, which comprises or consist of e.g. at least 10, 15, 20, 25, 30, 50, 100, 200, 500 or more consecutive nucleotides or amino acid residues of the longer molecule. Preferably, a fragment comprises or consists of less than 1000, 800, 600, 500, 300, 200, 100, 50, 30 or less consecutive nucleotides or amino acid residues of the longer molecule.

“Variants” refer to sequences which differ from natural sequences by one or more amino acid insertions, deletions or replacements, and are “substantially identical” to the native sequences as defined below.

The terms “protein” or “polypeptide” or “peptide” are used interchangeably and refer to molecules consisting of a chain of amino acids, without reference to a specific mode of action, size, 3-dimensional structure or origin. An isolated protein is a protein not found in its natural environment, such as a protein purified from a culture medium.

The term “identity”, “substantially identical”, “substantial identity” or “essentially similar” or “essential similarity” means that two polypeptides, when aligned pairwise using the Smith-Waterman algorithm with default parameters, comprise at least 60%, 70%, 80%, more preferably at least 90%, 95%, 96% or 97%, more preferably at least 98%, 99% or more amino acid sequence identity. Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA or using in EmbossWIN (e.g. version 2.10.0). For comparing sequence identity between two sequences, it is preferred that local alignment algorithms are used, such as the Smith Waterman algorithm (Smith T F, Waterman M S (1981) J. Mol. Biol. 147(1); 195-7), used e.g. in the EmbossWIN program “water”. Default parameters are gap opening penalty 10.0 and gap extension penalty 0.5, using the Blosum62 substitution matrix for proteins (Henikoff & Henikoff, 1992, PNAS 89, 915-919). In a preferred embodiment, the “identity”, “substantially identical”, “substantial identity” or “essentially similar” or “essential similarity” is assessed using the whole SEQ ID NO of a given polypeptide or nucleic acid molecule.

As used herein, the term “operably linked” refers to a linkage of elements (nucleic acid or protein or peptide) in a functional relationship. An element is “operably linked” when it is placed into a functional relationship with another element. For instance, a promoter or enhancer is operably linked to a coding sequence if it affects the transcription of the coding sequence. Operably linked means that the elements being linked are typically contiguous and, where necessary to join two protein coding regions, contiguous and in reading frame.

Expression will be understood to include any step involved in the production of a protein including, but not limited to transcription, post-transcriptional modification, translation, post-translational modification and secretion.

Nucleic acid construct is defined as a nucleic acid molecule, which is isolated from a naturally occurring gene or which has been modified to contain segments of nucleic acid which are combined or juxtaposed in a manner which would not otherwise exist in nature.

Control sequence is defined herein to include all components, which are necessary or advantageous for the expression of a recombinant protein. At a minimum, the control sequences include a promoter and transcriptional and translational stop signals.

### Cross-Linking

Cross-linking may be chemical cross-linking. In case of chemical cross-linking, the used (recombinant) protein is for example provided with a (chemical) linker and subsequently subjected to a linking reaction. The invention therefore provides a controlled release composition comprising a chemically cross-linked recombinant protein (preferably a gelatine-like protein) and a protein of interest, wherein the ratio of the average mesh size (s) of the recombinant protein and the average hydrodynamic radius of said protein of interest is smaller than 2, preferably smaller than 1.5, wherein said recombinant protein is chemically modified with a cross-linkable group.

Said cross-linkable group may be selected from, but is not limited to epoxy compounds, oxetane derivatives, lactone derivatives, oxazoline derivatives, cyclic siloxanes, or ethenically unsaturated compounds such as acrylates, methacrylates, polyene-polythiols, vinylethers, vinylamides, vinylamines, allyl ethers, allylesters, allylamines, maleic acid derivatives, itacoic acid derivatives, polybutadienes and styrenes.

Preferably as the crosslinkable group (meth)acrylates are used, such as alkyl-(meth)acrylates, polyester-(meth)acrylates, urethane-(meth)acrylates, polyether(meth)acrylates, epoxy-(meth)acrylates, polybutadiene-(meth)acrylates, silicone (meth)acrylates, melamine-Imethjacrylates, phosphazene-(meth)acrylates, (methlacrylamides and combinations thereof because of their high reactivity. Even more preferably said cross-linkable group is a methacrylate and hence the invention also provides methacrylated recombinant protein. Such a methacrylated recombinant protein is very useful in the preparation of a controlled release composition. Generally, the linking groups (for example methacrylate) are coupled to the recombinant protein and cross-linking is obtained by redox polymerisation (for example by subjection to a chemical initiator such as the combination potassium peroxodisulfate (KPS)/N,N,N′,N′ tetramethylethyenediamine (TEMED>> or by radical polymerisation in the presence of an initiator for instance by thermal reaction of by radiation such as UV-light).

Photo-initiators may be used in accordance with the present

invention and can be mixed into the solution of the recombinant protein. Photoinitiators are usually required when the mixture is cured by UV or visible light radiation. Suitable photo-initiators are those known in the art such as radical type, cation type or anion type photo-initiators.

Examples of radical type I photo-initiators are ahydroxyalkylketones, such as 2-hydroxy-1-[4-(2-hydroxyethoxy)phenyl]-2-methyl-Lpropanone (Irgacure™ 2.95.9: Ciba), 1-hydroxy-cyclohexylphenylketone (Irgacure™ 184: Ciba), 2-hydroxy-2-methyl-1-phenyl-1-propanone (Sarcure™ SR1173: Sartomer), oligo[2-hydroxy-2-methyl-1-{4-(1-methylvinyl)phenyl}propanone] (Sarcure™ SR1130: Sartomer), 2-hydroxy-2methyl-1-(4-tert-butyl-)phenylpropan-1-one, 2-hydroxy-[4′-(2hydroxypropoxy)phenyl]-2-methylpropan-1-one, 1-(4-Isopropylphenyl)-2hydroxy-2-methyl-propanone (Darcure™ 1116: Ciba); aminoalkylphenones such as 2-benzyl-2-(dimethylamino)-4′-morpholinobutyrophenone (Irgacure™ 16369: Ciba), 2-methyl-4′-(methylthio)-2-morpholinopropiophenone (Irgacure™ 907: Ciba); α,α-dialkoxyacetophenones such as α.,α-dimethoxy-α.phenylacetophenone (Irgacure™ 651: Ciba), 2,2-diethyoxy-1,2diphenylethanone (Uvatone™ 8302: Upjohn), α,α-diethoxyacetophenone (DEAP: Rahn), α,α-di-(n-butoxy)acetophenone (Uvatone™ 8301: Upjohn); phenylglyoxolates such as methylbenzoylformate (Darocure” MBF: Ciba); benzoin derivatives such as benzoin (Esacure™ BO: Lamberti), benzoin alkylethers (ethyl, isopropyl, n-butyl, iso-butyl, etc.), benzylbenzoin benzyl ethers, Anisoin; mono- and bis-Acylphosphine oxides, such as 2,4,6-trimethylbenzoyl-diphenylphosphine oxide (Lucirin™ TPO: BASF), ethyl-2,4,6-trimethylbenzoylphenylphosphinate (Lucirin™ TPO-L: BASF), bis(2,4,6-trimethylbenzoyl)-phenylphosphine oxide (Irgacure™ 819: Ciba), bis(2,6-dimethoxybenzoyl)-2,4,4-trimethyl-pentylphosphineoxide (Irgacure™ 1800 or 1870). Other commercially available photo-initiators are 144-(phenylthio)-2-(O-benzoyloxime)]-1,2-octanedione (Irgacure™ OXE01), 1-[9-ethyl-6-(2-methylbenzoyl)-9H-carbazol-3-yl]-1-(O-acetyloxime)ethanone (Irgacure OXE02), 2-hydroxy-1-{4-[4-(2-hydroxy-2-methyl-propionyl)-benzyl]-phenyl}-2-methyl-propan-Lone (Irgacure127), oxy-phenyl-acetic acid 2-[2 oxo-z-phenylacetoxy-ethoxyj-ethyl ester (Irgacure754), oxy-phenyl-acetic-2-[2-hydroxy-ethoxyj-ethyl ester (Irgacure754), 2-(dimethylamino)-2-(4-methylbenzyl)-1-[4(4-morpholinyl) phenylj-Lbutanone (Irgacure 379), 1-[4-[4-benzoylphenyl)thio]phenyl]-2-methyl-2-[(4-methylphenyl)sulfonyl)]-Ipropanone (Esacure 1001M from Lamberti), 2,2′-bis(2-chlorophenyl)-4,4′,5,5′tetraphenyl-1,2′-bisimidazole (Omnirad BCIM from 10M).

Examples of type II photo-initiators are benzophenone derivatives such as benzophenone (Additol™ BP: DCB), 4-hydroxybenzophenone, 3hydroxybenzophenone, 4,4′-dihydroxybenzophenone, 2,4,6-trimethylbenzophenone, 2-methylbenzophenone, 3-methylbenzophenone, 4-methylbenzophenone, 2,5-dimethylbenzophenone, 3,4-dimethylbenzophenone, 4-(dimethylamino)benzophenone, [4-(4-methylphenylthio)phenyl]phenyl-17-methanone, 3,3′-dimethyl-4-methoxy benzophenone, methyl-2-benzoylbenzoate, 4-phenylbenzophenone, 4,4-bis(dimethylamino)benzophenone, 4,4-bis(diethylamino)benzophenone, 4,4bis(ethylmethylamino)benzophenone, 4-benzoyl-N,N,N trimethylbenzenemethanaminium chloride, 2-5 hydroxy-3-(4-benzoylphenoxy)-N,N,N-trimethyl-1-propanamium chloride, 2-(Acryloyloxy)ethyl 4-(4-chlorobenzoyl)benzoate (Uvecryl™ P36: UCB), 4-benzoyl-N,N-dimethyl-N [2-(1-oxo-2-propenyl)oy]ethylbenzenemethanaminium chloride, 4-benzoyl-4′-methyldiphenyl sulphide, anthraquinone, ethylanthraquinone, anthraquinone-2-sulfonic acid sodium, salt, dibenzosuberenone; acetophenone derivatives such as acetophenone, 4′phenoxyacetophenone, 4′-hydroxyacetophenone, 3′hydroxyacetophenone, 3′ethoxyacetophenone; thioxanthenone derivatives such as thioxanthenone, 2-chlorothioxanthenone, 4-chlorothioxanthenone, isopropylthioxanthenone, 4isopropylthioxanthenone, 2,4-dimethylthioxanthenone, 2,4-diethylthioxanthenone, 2-hydroxy-3-(3,4-dimethyl-9-oxo-9H-thioxanthon-2yloxy)-N,N,N-trimethyl-1-propanaminium chloride (Kayacure QTX: Nippon Kayaku); diones such as benzyl, camphorquinone, 4,4′-dimethylbenzyl, phenanthrenequinone, phenylpropanedione; dimethylanilines such as 4,4′,4″methylidyne-tris(N,N-dimethylaniline) (Omnirad™ LCV from IGM); imidazole derivatives such as 2,2′-bis(2-chlorophenyl)-4,4′,5,5′-tetraphenyl-4,2′bisimidazole: titanocenes such as bis(eta-5-2,4-cyclopentadiene-1-yl)-bis-[2,6-difluoro-3-1H-pyrrol-1-yl]phenyl]titanium (Irgacure1′M784: Ciba); iodonium salt such as iodonium, (4 methylphenyl)-[4-(2-methylpropyl-phenyl)hexafluorophosphate (1-). If desired combinations of photo-initiators may also be used.

For acrylates, diacrylates, triacrylates or multifunctional acrylates, type I photo-initiators are preferred. Especially alpha-hydroxyalkylphenones, such as 2-hydroxy-2 methyl-1-phenyl propan-1-one, 2-hydroxy-2-methyl-1-(4tert-butyl-) phenylpropan-1-one, 2-hydroxy-[4′-(2-hydroxypropoxy)phenyl]-2-methylpropan-1-one, 2-hydroxy-I [4-(2-hydroxyethoxy)phenyl]-2-methyl18propan-1-one, I hydroxycyclohexylphenylketone and oligo[2-hydroxy-2-methyl]-{4-(1 methylvinyl)phenyl}propanone], alpha-aminoalkylphenones, alphasulfonylalkylphenones and acylphosphine oxides such as 2,4,6-trimethylbenzoyl-diphenylphosphine oxide, ethyl-2,4,6-trimethylbenzoyl-phenylphosphinate and bis(2,4,6-trimethylbenzoyl)-phenylphosphine oxide, are preferred.

Cross-linking by infrared radiation is also known as thermal curing.

Thus cross-linking may be effectuated by combining the ethylenically unsaturated groups with a free radical initiator and optionally a catalyst and heating the mixture. Exemplary free radical initiators are organic peroxides such as ethyl peroxide and benzyl peroxide; hydroperoxides such as methyl hydroperoxide, acyloins such as benzoin; certain azo compounds such as α,α′azobisisobutyronitrilo and y,y′-azobis(y-cyanovaleric acid); persulfates; peroxosulfates; peracetates such as methyl peracetate and tert-butyl peracetate; peroxalates such as dimethyl peroxalate and dj(tert-butyl) peroxalate: disulfides such as dimethyl thiuram disulfide and ketone peroxides such as methyl ethyl ketone peroxide. Temperatures in the range of from about 23° C. to about 150° C. or from 23° C. to 150° C. are generally employed. More often, temperatures in the range of from about 37° C. to about 110° C. or from 37° C. to 110° C. are used. When selecting a cross-linking method it is of high importance to verify that the protein of interest is not ‘damaged’ by the reaction and maintains its therapeutic activity.

The use of methacrylated recombinant protein is especially preferred in combination with a protein of interest, because cross-linking of a methacrylated recombinant protein such as a gelatine-like protein can be performed in the presence of a protein of interest without cross-linking the protein of interest.

As a result of cross-linking, a controlled release composition comprising a pharmaceutical, i.e. a protein of interest is obtained. The mesh size or pore size of the obtained product is dependent on the used recombinant protein and the cross-linking density. The mesh size is defined as the average distance between two neighbouring cross-links in the hydrogels polymer network. If a protein of interest is used as a pharmaceutical, the mesh size can be both larger and smaller than the hydrodynamic radius of the protein of interest. The hydrodynamic radius RH is the apparent radius of a protein of interest in the matrix comprising a recombinant protein taken into account all environmental effects. As such the hydrodynamic radius is derived from the diffusion coefficient D via the relation D=kT/61tTJRH, in which k is Boltzmann's constant, T is the temperature in Kelvin, 1 C is 3.14, and 11 is the viscosity of the solution in mPa·s. In the current invention the hydrodynamic radius is preferably measured at physiological conditions. The speed of degradation of the obtained product depends on the amount of cross-links: the more cross-links the slower the degradation. In a preferred embodiment, the speed of degradation is within one year. As release profiles of pharmaceuticals usually extend to a couple of weeks or maximally a few months it is preferable that the matrix consisting of a recombinant protein degrades in a similar time window. The final charge density of the obtained product depends both on the used amino acid sequence of the recombinant protein as well as on the degree of cross-linking. The obtained product can have different appearances, for example dense/homogenous or macroporous.

The release profile of the used pharmaceutical, i.e. protein of interest can be from several hours (diffusion controlled) to weeks or months (controlled by degradation speed). A combination of both mechanisms can also occur. For most applications a slow release is preferred and hence biodegradation as main mechanism.

As described, the cross-linking can be obtained by cross-linking (meth)acrylate residues introduced in the pre-modification of the recombinant protein. However, it is also possible to use a chemical cross-linker that does not need a separate coupling to the used recombinant protein. In another embodiment, the invention provides a method for preparing a controlled release composition comprising the steps of:
providing a solution of a recombinant protein, preferably a gelatine-like protein and a pharmaceutical, protein of interest
cross-linking said recombinant protein to obtain a three dimensional structure, wherein said cross-linking is obtained by using a chemical crosslinker selected from water soluble carbodiimide, non-soluble carbodiimide, dialdehyde di-isocyanate, aldehyde compounds such as formaldehyde and glutaraldehyde, ketone compounds such as diacetyl and chloropentanedion, bis(2-chloroethylurea), 2-hydroxy-4,6-dichloro-L, 3,5-triazine, reactive halogen containing compounds disclosed in U.S. Pat. No. 3,288,775, carbamoyl pyridinium compounds in which the pyridine ring carries a sulphate or an alkyl sulphate group disclosed in U.S. Pat. No. 4,063,952 and U.S. Pat. No. 5,529,892, divinylsulfones, and the like. S-triazine derivatives such as 2-hydroxy-4,6-dichloro-s-triazine are well known cross-linking compounds.

Basically the cross-linking occurs between two reactive groups on different gelatin molecules. Particularly preferred is the use of water soluble carbodiimide 1-(3-Dimethylaminopropyl)-3-ethylcarbodiimide hydrochloride. Depending on the type of recombinant protein (the number of cross-linkable groups) and the method of cross-linking selected a certain cross-link density can be obtained which is strongly related to the average mesh size that can be achieved. When a cross-linking group is coupled to the gelatin in a separate step and for the hydrogel dense structure is desired it is preferred that at 20%, or at least 50% of the cross-linkable groups in the gelatin are activated, more preferably at least 75%. Most preferably the degree of substitution is close to 100%.

In addition, reference to an element by the indefinite article “a” or “an” does not exclude the possibility that more than one of the element is present, unless the context clearly requires that there be one and only one of the elements. The indefinite article “a” or “an” thus usually means “at least one”. The term “comprising” is to be interpreted as specifying the presence of the stated parts, steps or components, but does not exclude the presence of one or more additional parts, steps or components. In addition the verb “to consist” may be replaced by “to consist essentially of” meaning that a polypeptide or a nucleic acid construct or a composition or a cell as defined herein may comprise additional component(s) than the ones specifically identified, said additional component(s) not altering the unique characteristic of the invention.

Each embodiment as identified herein may be combined together unless otherwise indicated. All patent and literature references cited in the present specification are hereby incorporated by reference in their entirety.

The present invention is further illustrated by the following examples, which should not be construed for limiting the scope of the invention.

### EXAMPLES

SEQ ID No. 2: represents a monomer of HBC (Heparin Binding Collagen like polypeptide)

DMGIKGDRGEIGPPGPRGEDGPEGPKGRGGPNGDPGPLGPGEKGKLGVPG
LPGYPGRQGPKGSIGFPGFPGANGEKGGRGTPGKPGPRG
QRGPTGPRGERGPRGITGKPGPKGNSGGDGPAGPPGERGPNGPQGPTGFP
GPKGPPGPPGKDGLPGMKGHRGFRGETGFQGKTGPPGPPGVVGPQGLPGM
KGHRGFMGERGHPGPPGPPGEQGLPG

The sequence above is based on the sequence of human Type V collagen alpha 1 chain (Col5a1) position 821-1030. Type V collagen alpha1 chain binds to heparin/heparan sulphate. Especially the so called HepV portion (in bold) that binds to heparin strongly. (HBC)n is an example of a preferred recombinant protein which has been enriched in heparin binding sites. The chosen heparin binding site is “GLPGMKGHRGFS” (in bold in SEQ ID NO:2 above). Actually, S was omitted, since it is a glycosylation site. Where needed Ts and Ss were substituted to A to avoid the glycosylation

### Example 1

An heparin binding gelatine was produced based on a nucleic acid sequence that encodes for a part of the gelatine amino acid sequence of human COL5a1 and modifying this nucleic acid sequence. The methods as disclosed in EP-A-0926543, EP-A-1014176 and WO01/34646 were used. This heparin binding gelatine is named HBC and the sequence of this heparin binding gelatine according to the invention is given in SEQ ID NO: 2. Via standard subcloning methods multimers of the HBC monomer have been prepared: (HBC)n with n being 4, 8, or 12.

### Example 2

### BMP2 Delivery from a Matrix of Heparine Binding Recombinant Gelatin

It has been demonstrated that bone morphogenetic factor-2 (BMP-2) and fibroblast growth factor 2 (FGF-2) bound to the heparine binding recombinant gelatin matrix, in the presence of heparin is able to control the release of the growth-factor and when this matrix is implanted subcutaneously ectopic bone is formed. While this procedure is different from bone formation within a bony defect, it does provide a suitable method for testing the ability to control the release of a bioactive growth factor (BMP-2 or FGF-2) in vivo. Two methods for preparing such a matrix are provided as example.

### Method 1: Methylacrylated Gelatine Matrix

Recombinant. gelatin-like proteins (HBC)4 and P4 were derivatized with methacrylate residues as follows. 2.5 g gelatin was dissolved in 200 ml phosphate buffer of pH 7.4. Solutions under a nitrogen atmosphere were heated to 50° C. and methacrylic-anhydride (MA-Anh) was added. To achieve different degrees of substitution, the MA-Anh:gelatin ratio was varied. During the methacrylation reaction, the pH of the solution was regularly controlled and, if necessary, kept between 7 and 7.4 by the addition of 1 M NaOH solution. After vigorous stirring at 50° C. for one hour, the solutions were extensively dialyzed against water (dialysis tubes with 14 kDa MWCO Medicell International, London, UK). Dried products were obtained by lyophilization and were stored in sealed glass containers at 4° C.

The two growth factors BMP-2 and FGF-2 were tested. Gels were polymerized in the presence of heparin and one of the above growth-factors in ratios of heparin to growth factor of 1:1 and 40:1. Recombinant gelatine matrices including one of two growth factors tested and heparin were prepared as follows. Hydrogels with an initial gelatin concentrations of 5, 10, 15, 20, 25, 30 and 40% (w/w) were prepared. Methacrylated gelatin was dissolved in phosphate buffer of pH 7.4 containing 0.05% NaN3, and solutions were centrifuged (5 min, 10000 RPM). Upon centrifugation, 596 mg gelatin solution was filled in an Eppendorf tube, and 75 μl phosphate buffer of pH 7.4 (or protein stock solution for release experiments) were added and gently mixed. KPS 20 mg/ml stock solution (56.5 μl) and TEMED 20% stock solution (22.5 μl) were added and mixed to induce cross-linking of the gelatin methacrylate residues. The solution was used to fill 1 ml syringes (Becton-Dickinson, Franklin Lake, N.J.). After 1.5 h, the syringes were opened to remove the hydrogels, which were cut into cylinders of 6 mm length and 2.3 mm radius.

### Method 2: Chemical crosslinking using 1-ethyl-3-(3-dimethylaminopropyl) carbodiimide hydrochloride (EDC)

The growth factors BMP-2 and FGF-2 can both bind heparin and will bind to a (HBC)4 recombinant gelatin matrix in the presence of heparin. Hydrogel matrices were prepared up front and loaded with heparin and one of the two tested growth-factors in ratios of heparin to growth factor of 1:1 and 40:1 by diffusion. Recombinant gelatin (HBC)4 was used to test the heparine binding recombinant gelatine controlled release system. Control gels of recombinant gelatine P4 (as disclosed in EP 1 014 176) lacking the heparin binding capacity were loaded with equivalent amounts of the growth-factors, and heparin. Gelatine hydrogels of HBC and P4 were prepared as a 25% w/w solution in water at room temperature. 60 μl of 25% EDC was added to 400 μl 25% P4 and mixed. The solution was used to fill 1 ml syringes (Becton-Dickinson, Franklin Lake, N.J.). After 1.5 h, the syringes were opened to remove the hydrogels, which were cut into cylinders of 6 mm length and 2.3 mm radius.

### Ectopic Bone Formation Assay

Gels were implanted subcutaneously into rats and were allowed to remain for two weeks. When the matrices were extracted little to no bone was observed from the gels that did not contain the heparin binding release system while the gels that did contain the system showed significant bone formation as shown in Table 3.

| | Ectopic |
|---|---|
| Treatment | bone formation |
| P4 + 20 microgram BMP-2 | + |
| HBC + 26 microgram heparin + 20 | +++ |
| microgram BMP-2 | |
| HBC + 1 mg heparin + 20 microgram | ++++ |
| BMP-2 | |
| HBC + 26 mg heparin + 20 microgram | ++ |
| FGF-2 | |
| HBC + 1 mg heparin + 20 microgram | +++ |
| FGF-2 | |

The release system enhanced the formation of ectopic bone within the matrix, demonstrating the viability of the release system in vivo.

1.-12. (canceled)

13. A recombinant gelatin-like protein essentially free of hydroxyproline residues enriched in a heparin binding site and/or a heparan sulfate binding site, wherein the heparin binding site and/or a heparan sulfate binding site is selected from the group consisting of: GBBGB (SEQ ID NO: 11), GLPGMKGHRGFS (SEQ ID NO: 12), GRKGR (SEQ ID NO: 13), GKRGK (SEQ ID NO: 17) and KEDK (SEQ ID NO: 14), wherein B is a basic amino acid.

14. A recombinant gelatin-like protein according to claim 13, wherein the recombinant protein is essentially free of glycosylation.

15. A recombinant gelatin-like protein according to claim 13 which has a molecular weight of at least about 15 kDa.

16. A recombinant gelatin-like protein according to claim 13, wherein the recombinant protein is a multimeric peptide that comprises at least 2, 3, 4, 5, 6, 7, 8, 9 or 10, 11, 12 repeats of a monomer.

17. A recombinant gelatin-like protein according to claim 16, wherein the monomer comprises at least one XRGD, wherein X is not D or P or O.

18. A recombinant gelatin-like protein according to claim 13 which is a methacrylated recombinant protein.

19. A composition comprising the recombinant gelatin-like protein as defined in claim 13 and a heparin or heparan sulfate.

20. A composition according to claim 19, wherein the heparin and/or heparin sulphate is from a non-mammalian source.

21. A matrix comprising the composition of claim 19 which is a three dimensional network comprising biocompatible polymers which are covalently or non-covalently cross linked.

22. A matrix according to claim 21 wherein the biocompatible polymers comprise a recombinant gelatin-like protein essentially free of hydroxyproline residues enriched in a heparin binding site and/or a heparan sulfate binding site, wherein the heparin binding site and/or a heparan sulfate binding site is selected from the group consisting of: GBBGB (SEQ ID NO: 11), GLPGMKGHRGFS (SEQ ID NO: 12), GRKGR (SEQ ID NO: 13), GKRGK (SEQ ID NO: 17) and KEDK (SEQ ID NO: 14), wherein B is a basic amino acid.

23. A controlled release system for a protein of interest and/or a cell comprising a matrix according to claim 21 and further comprising a protein of interest and/or a cell.

24. A controlled release system according to claim 23 wherein the protein of interest and/or cell comprises a heparin and/or heparan sulfate binding site.

25. A cell support system comprising a matrix according to claim 21.

26. A method for producing a recombinant gelatin-like protein comprising:
preparing an expression vector comprising a nucleic acid sequence encoding a recombinant protein according to claim 13,
expressing said nucleic acid sequence in a yeast,
culturing said yeast under suitable fermentation conditions to allow expression of said nucleic acid sequence;
optionally purifying said recombinant protein from the culture.

27. A controlled release system as described in claim 23 for use in promoting cell repair, regeneration or remodeling for cardiovascular disease, myocardial repair, angiogenesis, bone repair and regeneration, wound treatment, neural stimulation/therapy or diabetics.

28. A cell support system as described in claim 25 for use in promoting cell repair, regeneration or remodeling for cardiovascular disease, myocardial repair, angiogenesis, bone repair and regeneration, wound treatment, neural stimulation/therapy or diabetics.


SEQUENCE LISTING (17 sequences)

SEQ ID NO: 1 - protein, 14 aa, Artificial
  Synthetic sequence
KAFAKLAARLYRKA

SEQ ID NO: 2 - protein, 215 aa, Artificial
  Synthetic sequence
DMGIKGDRGEIGPPGPRGEDGPEGPKGRGGPNGDPGPLGPGEKGKLGVPGLPGYPGRQGPKGSIGFPGFPGANGEKGGRGTPGKPGPRGQRGPTGPRGERGPRGITGKPGPKGNSGGDGPAGPPGERGPNGPQGPTGFPGPKGPPGPPGKDGLPGMKGHRGFRGETGFQGKTGPPGPPGVVGPQGLPGMKGHRGFMGERGHPGPPGPPGEQGLPG

SEQ ID NO: 3 - protein, 14 aa, Artificial
  Synthetic sequence
KHKGRDVILKKDVR

SEQ ID NO: 4 - protein, 19 aa, Artificial
  Synthetic sequence
YEKPGSPPREVVPRPRPCV

SEQ ID NO: 5 - protein, 15 aa, Artificial
  Synthetic sequence
KNNQKSEPLIGRKKT

SEQ ID NO: 6 - protein, 6 aa, Artificial
  Synthetic sequence
KDPKRL

SEQ ID NO: 7 - protein, 6 aa, Artificial
  Synthetic sequence
YRSRKY

SEQ ID NO: 8 - protein, 6 aa, Artificial
  Synthetic sequence
YKKPKL

SEQ ID NO: 9 - protein, 7 aa, Artificial
  Synthetic sequence
AKRSSKM

SEQ ID NO: 10 - protein, 6 aa, Artificial
  Synthetic sequence
CRKRCN

SEQ ID NO: 11 - protein, 5 aa, Artificial
  Synthetic sequence
GBBGB

SEQ ID NO: 12 - protein, 12 aa, Artificial
  Synthetic sequence
GLPGMKGHRGFS

SEQ ID NO: 13 - protein, 5 aa, Artificial
  Synthetic sequence
GRKGR

SEQ ID NO: 14 - protein, 4 aa, Artificial
  Synthetic sequence
KEDK

SEQ ID NO: 15 - protein, 11 aa, Artificial
  Synthetic sequence
GLPGMKGHRGF

SEQ ID NO: 16 - protein, 8 aa, Artificial
  Synthetic sequence
YKKIIKKL

SEQ ID NO: 17 - protein, 5 aa, Artificial
  Synthetic sequence
GKRGK